(19) United States
(12) Patent Application Publication
(10) Pub. No.: US 2026/0179734 A1
(43) Pub. Date: Jun. 25, 2026

(54) SYSTEM AND METHOD FOR PREDICTING AND OPTIMIZING CLINICAL TRIAL OUTCOMES

(71) Applicant: VALO HEALTH, INC., Lexington, MA (US)

(72) Inventors: Alex Greenfield, Boston, MA (US); Olivia Sabik, Boston, MA (US); Kelsey Cadirov, Boston, MA (US); Leon Furchtgott, Boston, MA (US); Aaron Mackey, Boston, MA (US)

(21) Appl. No.: 19/127,620

(22) PCT Filed: Nov. 17, 2023
(86) PCT No.: PCT/US2023/080273

§ 371 Date: May 6, 2025

Related U.S. Application Data
(60) Provisional application No. 63/429,775, filed on Dec. 2, 2022.

(51) Int. Cl. G16H 10/20; G06N 7/01; G06N 20/20; G16H 15/00; G16H 50/70

(52) U.S. Cl. CPC G16H 10/20; G06N 7/01; G06N 20/20; G16H 15/00; G16H 50/70

(57) ABSTRACT

A vector associated with a clinical trial is obtained from one or more data sources. The vector comprises one or more fixed elements and one or more optimizable elements. A trial outcome predictor, trained on data related to a plurality of historical clinical trials, is used to determine a probabilistic model of an outcome of the clinical trial based on the vector. An explainability model is used to determine contribution scores for the vector based on the probabilistic model. Each contribution score is indicative of a relative contribution of an associated element of the vector to the outcome of the clinical trial. An explainable prediction of the trial outcome of the clinical trial is generated based on the probabilistic model and one or more of the contribution scores which are associated with the one or more optimizable elements. The explainable prediction is output for review by a user.

### RELATED APPLICATIONS

This application claims the benefit of U.S. Provisional Application No. 63/429,775 (filed on Dec. 2, 2022), which is incorporated by reference herein in its entirety.

### TECHNICAL FIELD

The present disclosure relates to the automated modelling of outcomes of future clinical trials. Particularly, but not exclusively, the present disclosure relates to predicting the probability of a future clinical trial achieving a trial outcome. Particularly, but not exclusively, the present disclosure relates to optimizing a configuration of a future clinical trial to improve the probability of the trial outcome being achieved.

### BACKGROUND

Approximately one in ten drug candidates successfully pass through clinical trial testing and regulatory approval. Accurately predicting outcomes of clinical trials therefore provides multiple opportunities in clinical development, including prioritizing drug development investment, modifying existing trial portfolios to maximize success, and finding undervalued molecules.

### SUMMARY OF DISCLOSURE

According to an aspect of the present disclosure, there is provided a system and method, and computing instructions configured for execution on one or more processors, for generating an explainable prediction of a trial outcome of a clinical trial. A trial configuration vector associated with the clinical trial is obtained from one or more data sources. The trial configuration vector comprises one or more fixed elements and one or more optimizable elements. A probabilistic model of an outcome of the clinical trial is determined using a trial outcome predictor based on the trial configuration vector. The trial outcome predictor has been trained on data related to a plurality of historical clinical trials. A plurality of contribution scores for the trial configuration vector are determined using an explainability model based on the probabilistic model. Each contribution score of the plurality of contribution scores is indicative of a relative contribution of an associated element of the trial configuration vector to the outcome of the clinical trial. An explainable prediction of the trial outcome of the clinical trial is generated based on the probabilistic model and one or more contribution scores of the plurality of contribution scores. The one or more contribution scores being associated with the one or more optimizable elements. The explainable prediction is output for review by a user.

According to a further aspect of the present disclosure, there is provided a system and method, and computing instructions configured for execution on one or more processors, for optimizing the parameters of a clinical trial. A first trial configuration associated with the clinical trial is obtained from one or more data sources. The first trial configuration comprises values associated with one or more fixed trial parameters and at least one optimizable trial parameter. An outcome predictor is obtained, where the outcome predictor estimates a relationship between a trial configuration of a clinical trial and an outcome of the clinical trial. The first trial configuration is optimized to improve an outcome of the clinical trial by determining, using the outcome predictor and the first trial configuration, an updated value of the at least one optimizable trial parameter such that a first estimated outcome of the clinical trial is greater than a second estimated outcome of the clinical trial, and creating an updated trial configuration comprising the updated value of the at least one optimizable trial parameter. The first estimated outcome is determined from the outcome predictor based on the updated trial configuration and the second estimated outcome is determined from the outcome predictor based on the first trial configuration. The updated trial configuration is output for review by a user.

In accordance with the above, and with the disclosure herein, the present disclosure includes improvements in computer functionality or in improvements to other technologies at least because the disclosure herein discloses an artificial intelligence (AI) based model, e.g., a trial outcome predictor, that is trained with data of a plurality of historical clinical trials, and where the trial outcome predictor, when deployed on the underlying system, allows the systems and methods of the present disclosure to execute with fewer iterations, and use fewer computing resources, than prior art related systems and methods. That is, the present disclosure describes improvements in the functioning of the computer itself or “any other technology or technical field” because the increased predictive improvement provided by the trial outcome predictor allows the underlying computer system to utilize less processing and memory resources compared to prior art systems and methods because the trial outcome predictor can generate or determine a probabilistic model, or otherwise result, of a clinical trial having a high likelihood of success using fewer compute cycles, or otherwise iterations, that has less of an impact on the underlying computing device compared to previous prior art systems and methods. Said another way, the systems and methods of the present disclosure improve over the prior art at least because prior art systems and methods require an empirical or trial-and-error approach that can involve real-world trials and/or data-entry that can result in, and require, large database and memory utilization and processor usage to arrive at a similar real-world or simulated trial outcome that has a same or similar highly accurate or predictive result. By contrast, the disclosed systems and methods describe generation and/or use of a trial configuration vector that defines a streamlined set of elements (e.g., fixed element and optimizable elements) that use a more limited, known set of data related to the elements, which require less memory usage and/or processing utilization compared to a conventional approach where large sets of unknown, potentially irrelevant data is used or required.

In addition, the present disclosure relates to improvement to other technologies or technical fields at least because the present disclosure discloses generation and/or use of an explainability model. The explainability model improves over conventional prior art AI-related models by providing technical clarity in the form a visual or data view (e.g., the trial outcome predictor) of the output or result of the disclosed AI-model. Said another way, conventional AI-models and related algorithms typically provide no clarity, view, or otherwise explanation, as to the generation of the model as to how the output or result is achieved. Such prior art methods operate as black-box computational structures that provide little or no insight to the model or how it was trained. Such prior art techniques can be detrimental in the training or generation of AI models because technical biases or errors can be implicitly built into such AI models, which can result in technical biases or errors in the output of the model that cannot be discovered, improved, or otherwise determined. By contrast, the explainability model of the present disclosure provides a view into the model (e.g., the trial outcome predictor) and its related output by providing a data-based and/or visual representation or otherwise explanations of how the training data impacts or otherwise determines the output of the disclosed AI model, e.g., the trial outcome predictor. Said another way, the explainability model allows for a window or view into how the AI model is currently trained and how such training impacts the output result. This allows for the AI-model to be retrained or reconfigured, e.g., with different training data, such as different trial configuration vectors having different fixed and/or optimizable parameters and/or with different data from additional data sources, in order to eliminate error and/or bias in a second version of an AI model, e.g., trial outcome predictor and its related output.

Still further, the present disclosure relates to improvement to other technologies or technical fields at least because the disclosed systems and methods provide normalization and/data formatting of data as received, ingested, and/or otherwise obtained from one or more data sources to create, generate, or otherwise obtain a trial configuration vector used for training an AI model, e.g., a trial outcome predictor as described herein. In particular, the data as received from various data sources may comprise data from different databases, data sinks, or otherwise data locations where such data may not be compatible (e.g., in a raw or otherwise as-received form). The systems and methods of the present disclosure may operate to normalize or format such data, e.g., to create a normalized set of data, for use in training trial outcome predictor as described herein.

In addition, the present disclosure relates to improvement to other technologies or technical fields at least because the disclosed systems and methods can reduce data sets and increase security by removing personably identifiable information (PII) from data received by data sources that include PII. PII may include sensitive data such as a person's health data. Such data reduction and/or normalization can increase security of the systems or methods described herein by eliminating data stored in memory and also reducing the risk of sensitive data security leaks at the same time.

The present disclosure includes specific features other than what is well-understood, routine, conventional activity in the field, and/or otherwise adds unconventional steps that confine the disclosure to a particular useful application, e.g., systems and methods for generating an explainable prediction of a trial outcome of a clinical trial and/or optimizing the parameters of a clinical trial.

Further features and aspects of the disclosure are provided in the appended claims.

### BRIEF DESCRIPTION OF DRAWINGS

The present disclosure will now be described by way of example only with reference to the accompanying drawings in which:

FIG. 1 shows a system for generating an explainable prediction of a trial outcome of a clinical trial according to embodiments of the present disclosure;

FIG. 2 illustrates an example trial configuration according to embodiments of the present disclosure;

FIGS. 3A and 3B show example contribution scores according to embodiments of the present disclosure;

FIG. 4 shows a portion of an example report according to embodiments of the present disclosure;

FIG. 5 shows a process for optimizing a trial configuration according to embodiments of the present disclosure;

FIGS. 6A and 6B show the results of predicting the outcomes of a plurality of clinical trials according to embodiments of the present disclosure.

FIGS. 7A and 7B show the predicted probability of success for two clinical trials according to embodiments of the present disclosure.

FIGS. 8A and 8B show a method for generating an explainable prediction of a trial outcome of a clinical trial according to embodiments of the present disclosure;

FIG. 9 shows a method for optimizing the parameters of a clinical trial according to embodiments of the present disclosure; and

FIG. 10 shows an example computing system for carrying out the methods of the present disclosure.

### DETAILED DESCRIPTION

The ability to predict the likely outcome of a clinical trial is an important step when trying to prioritize drug development investment, modify existing trial portfolios to maximize success, and find undervalued molecules. The clinical trial may be a proposed clinical trial—i.e., a clinical trial which has not yet begun and may still be in the design and development phase. Typically, the intervention has been identified but other factors such as sponsors, sponsor sites, and the like have yet to be confirmed. Predicting the likely outcome of such trials at this stage may help to avoid undertaking trials which are unlikely to succeed. This may help to avoid unnecessary patient and public involvement in trials which are likely to have limited public benefit. Alternatively, the clinical trial may have already begun but could nevertheless still be improved. Existing approaches to predicting clinical trial outcomes typically fail to predict, with high accuracy and mechanistic insight, the likelihood of trial success. Such approaches are inflexible and include only limited dimensions of data. Moreover, the basic algorithms utilized by such existing approaches are not able to construct different “what-if” scenarios for a clinical trial. The level of insight gained from such approaches is invariably limited as it is not possible for users to gain a clear understanding of the factors which are contributing to the likely success or failure of a clinical trial. Successfully and automatically optimizing clinical trial designs using existing approaches is thus a difficult and inefficient task.

The systems and methods of the present disclosure help to identify and quantify key sources of risks for assets and clinical trials by providing explainable estimates of the likely outcome of a clinical trial. Moreover, the present disclosure provides systems and methods for optimizing the configuration of a clinical trial. Such optimization helps to improve the likelihood of success for a clinical trial thereby helping to make more efficient use of resources by identifying aspects of the design of a clinical trial which can be modified prior to undertaking the clinical trial.

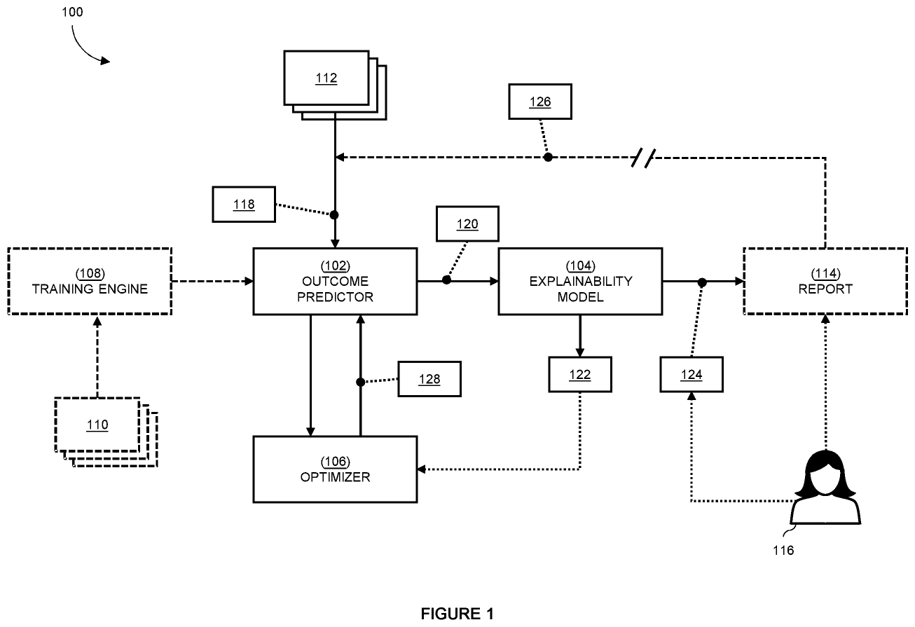

FIG. 1 shows a system 100 for generating an explainable prediction of a trial outcome of a clinical trial according to embodiments of the present disclosure.

The system 100 comprises a trial outcome predictor 102, an explainability model 104, and an optimizer 106. In embodiments, the system 100 further comprises a training unit 108 which trains a trial outcome predictor using historical clinical trial data 110. FIG. 1 further shows one or more data sources 112 in communication with the trial outcome predictor 102, and a report 114 which is viewable by a user 116. Also shown in FIG. 1 are a trial configuration vector 118 associated with a clinical trial, a probabilistic model 120 of an outcome of the clinical trial, a plurality of contribution scores 122, an explainable prediction 124 of the trial outcome, a first updated trial configuration vector 126, and a second updated trial configuration vector 128.

The clinical trial is described, or represented, by the trial configuration vector 118. The clinical trial may be a proposed clinical trial which has yet to begin or a clinical trial already being undertaken. The system 100 predicts a trial outcome for the clinical trial based on the feature values, or elements, within the trial configuration vector 118. The trial configuration vector 118 is obtained from the one or more data sources 112 and passed to the trial outcome predictor 102. The trial configuration vector 118 comprises one or more fixed elements associated with one or more fixed trial features, and one or more optimizable elements associated one or more optimizable trial features. The trial outcome predictor 102, which has been trained on data related to a plurality of historical clinical trials, determines the probabilistic model 120 of the outcome of the clinical trial based on the trial configuration vector 118. In one embodiment, the trial outcome predictor 102 is trained by the training unit 108 using the historical clinical trial data 110. The explainability model 104 determines the plurality of contribution scores 122 for the trial configuration vector 118 based on the probabilistic model 120. Each contribution score of the plurality of contribution scores 122 is indicative of a relative contribution of an associated element of the trial configuration vector 118 to the outcome of the clinical trial. The explainable prediction 124 of the trial outcome of the clinical trial is generated based on the probabilistic model 120 and one or more contribution scores of the plurality of contribution scores 122. The one or more contribution scores from which the explainable prediction 124 is generated are associated with the one or more optimizable elements of the trial configuration vector 118. The explainable prediction 124 is output for review by the user 116. In one embodiment, the explainable prediction 124 is included in the report 114 which is output for review by the user 116.

The explainable prediction 124 provides insight into the factors which contribute to the outcome (e.g., success or failure) of the clinical trial. The insight provided by the explainable prediction 124 may help drive the creation of an improved configuration (design) of the clinical trial which in turn improves the likelihood of the trial outcome being met. The system 100 therefore allows the user 116 to investigate different “what-if” scenarios regarding the clinical trial in an efficient manner. The output of the system 100 also helps quantify the different success and risk factors of a clinical trial thereby helping to influence the decision making process when determining whether to conduct the clinical trial. The greater insight provided by the present disclosure thus helps develop improved clinical trials with improved chances of success and reduced risk. In addition, the present disclosure provides a greater level of explainability and understanding of the relationship between the configuration of the clinical trial and the predicted trial outcome. This in turn may help improve understanding and optimization of the clinical trial.

The trial configuration vector 118, alternatively referred to as a trial configuration or a trial vector, corresponds to a configuration of the clinical trial. That is, the trial configuration vector 118 encodes aspects related to the design and protocol of the clinical trial. Each element, or value, of the trial configuration vector 118 is associated with a feature of the clinical trial and may be binary valued, integer valued, or real valued. For example, elements associated with a categorical sponsor type feature may be encoded using an approach such as one-hot encoding to represent the different possible values for this feature (e.g., “industry” or “academia”), whereas an element associated with a feature corresponding to the number of investigators involved in the clinical trial may take a non-zero integer or real value. In embodiments, elements associated with non-binary numerical features may be transformed (e.g., normalized, log-transformed, etc.) prior to being passed to the trial outcome predictor 102.

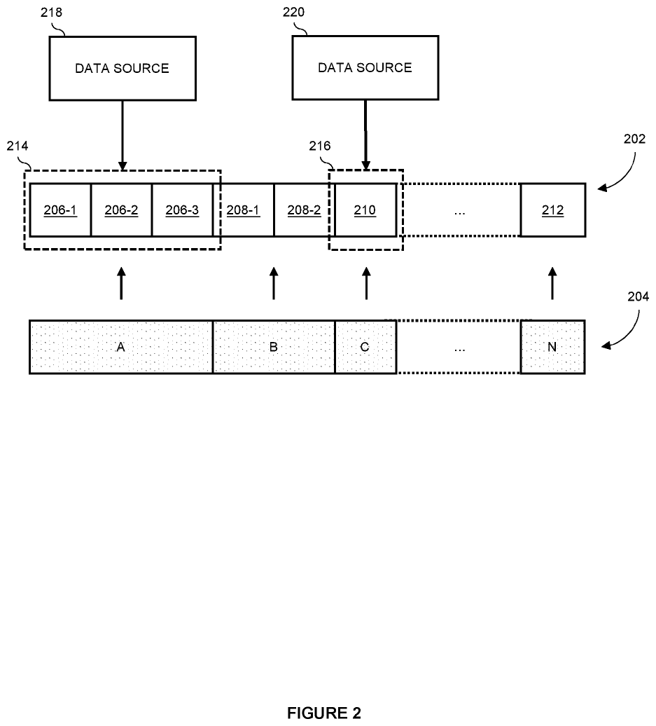

FIG. 2 illustrates an example trial configuration according to embodiments of the present disclosure.

FIG. 2 shows a trial configuration vector 202 associated with a plurality of features 204 of a clinical trial. The trial configuration vector 202 comprises elements 206-1, 206-2, 206-3, 208-1, 208-2, 210, and 212. A first element group 214 comprises elements 206-1, 206-2, 206-3 which are associated with feature “A” in the plurality of features 204. A second element group 216 comprises element 210 and is associated with feature “C” in the plurality of features 204. The first element group 214 is obtained from a first data source 218 and the second element group 216 is obtained from a second data source 220. The remaining elements in the trial configuration vector 202 may be obtained from the first data source 218, the second date source 220, or a number of other (not shown) data sources. The data as received from various data sources may be obtained, be received, or may otherwise comprise data from different databases or data sinks and may not be compatible in a raw or otherwise as-received form. The systems and methods of the present disclosure can operate to normalize or format such data, e.g., to create a normalized set of data, e.g., for use in populating elements of a trial configuration vector and/or for use in training a trial outcome predictor. In addition, for example in some aspects, data may be reduced, and security of the system increased, by removing personably identifiable information (PII) from data received by data sources that include PII. PII may include sensitive data such as a person's health data. Such data reduction and/or normalization can increase security of the systems or methods described herein by eliminating data stored in memory and also reducing the risk of sensitive data security leaks at the same time.

In the example shown in FIG. 2, feature “A” corresponds to an encoded feature (e.g., one-hot encoded) used to represent a categorical feature value. For example, feature “A” may correspond to a drug target type which can take one of three values: “enzyme”, “receptor”, or “ion channel”. The elements 206-1, 206-2, 206-3 within the first element group 214 may be binary valued and indicate which of the three drug target types the clinical trial relates to. For example, elements [1, 0, 0] may indicate an enzyme drug target type whilst elements [0, 0, 1] may indicate an ion channel drug target type.

Feature “C” within the example of FIG. 2 corresponds to a numerical feature. For example, feature “C” may correspond to the number of positive trials that the sponsor of the clinical trial has. As such, the element 210 within the trial configuration vector 202 which is associated with feature “C” may take a non-zero integer value.

As shown in FIG. 2, a trial configuration vector represents multiple features related to a clinical trial. A trial configuration vector, such as the trial configuration vector 202, thus comprises a concatenation of elements associated with features of the clinical trial. The concentration of elements for use by the trial configuration vector allows for a streamlined set of elements (e.g., fixed element and optimizable elements) that define a more limited, known set of data related to the elements, which require less memory usage and/or processing utilization compared to a conventional approach where large sets of unknown, potentially irrelevant data is used or required. Consequently, the plurality of features associated with a trial configuration vector (such as the plurality of features 204) may comprise a combination of features from different categories or groups of features, such as: biological features; chemical features; design and operation features which may include geographical features, sponsor features, and investigator features; keyword features; and miscellaneous features. Each of these categories of features may be understood as being associated with elements corresponding to separate vectors within the trial configuration vector 202. Incorporating data from such varied sources provides a greater variation of clinical trials to be represented. This increased representative capacity helps improve predictive accuracy.

The plurality of features associated with a trial configuration vector may comprise biological features related to a target associated with a clinical trial. The biological features may include mechanism of action features. Mechanism of action features seek to quantify the various mechanisms of action of the drug to which the clinical trial is directed (e.g., angiogenesis inhibitor, immunostimulant, tubulin inhibitor, and the like). The mechanism of action features may be represented by an n-dimensional vector, where n corresponds to the number of different mechanisms of action that may be represented by the system. As such, the elements of a trial configuration vector associated with a mechanism of action feature may be encoded using a categorical encoding approach such as one-hot encoding, dummy encoding, effect encoding, hash encoding, and the like. In embodiments, the biological features may include hierarchical mechanism of action features. Such features expand the above mechanism of action encoding to include higher-order groupings. That is, instead of encoding only master names for the mechanism of action for the drug of the clinical trial, names from each hierarchical level of the mechanism of action are encoded. For example, consider the anti-dopaminergic mechanism of action dopamine D2 receptor antagonist. Using a master name encoding approach (as described above), a dopamine D2 receptor antagonist could be represented by a single binary indicator value within the trial configuration vector. A hierarchical representation of this mechanism of action would split the master name (“dopamine D2 receptor antagonist”) into a first level “dopamine”, a second level “D2 receptor”, and a third level “antagonist”. Each level could then be represented by indicator values within the trial configuration vector. In such a hierarchical representation, a dopamine D3 receptor agonist would be represented in the same first and second levels (“dopamine” and “D3 receptor”) but a different third level (“agonist”). The hierarchical representation therefore allows for the similarity between different mechanisms of action to be quantified more accurately. This in turn helps improve the explainability of the model by providing a fine grained representation of the mechanism of action which is more likely to lead to the contribution of the different hierarchies of the mechanism of action being identified.

The chemical features are related to the target associated with a clinical trial. The chemical features may comprise chemical structure data related to the target drug. For example, the chemical structure data may include a vectorized representation of the SMILES string of the target. The chemical structure data may also include a vector representative of the molecular data related to the target such as molecular weight and categorically encoded molecule type (e.g., using one-hot encoding, dummy encoding, effect encoding, hash encoding, and the like). The chemical structure data may comprise an indicator variable representative of the presence or absence of chemical structure data for the target.

The design and operation features are associated with aspects such as the design, protocol, and operation of a clinical trial. The design and operation features may include geographical features, sponsor features, and/or investigator features. The geographical features may be included in a vector representing the trial country (or countries) and the trial region (or regions) within which the clinical trial is to take place or has taken place. For example, a clinical trial taking place in Germany, Canada, and the United Kingdom would have a categorically encoded geographical feature vector indicating the three countries associated with the trial and the trial regions of Europe and North America. The sponsor features include features related to the sponsor or sponsors of the clinical trial such as the number of sponsors, the sponsor types (e.g., government, pharmaceutical manufacturer, contract research organization, etc.), and the experience of the sponsors. The sponsor experience comprises the number of previous trials involving the sponsor which either completed, terminated, had a positive outcome in Phase I/II/III, or had a negative outcome in Phase I/II/III. The investigator features may include data relating to the investigators involved in the clinical trial such as the number of investigators, and the experience of the investigators.

The keyword features are related to study keywords associated with a clinical trial. The keyword features may include an encoded representation associated with study keywords such as “randomized”, “open label”, “pharmacodynamics”, etc. Example encoding approaches include one-hot encoding, dummy encoding, effect encoding, hash encoding, and the like. The keyword features may also include an encoded representation associated with notes associated with the clinical trial such as “expanded indication”, “expanded access”, “investigator initiated”, and the like. The keyword features may also include an encoded representation of medical subject heading (MeSH) terms.

The miscellaneous features are related to various aspects of a clinical trial not covered by the above feature groupings. For example, the route of administration (e.g., injectable, inhaled, topical, etc.), the drug origin (e.g., chemical, biologic, etc.), or the therapeutic area (e.g., oncology, autoimmune, etc.). In all such examples, the categorical features may be encoded using a suitable categorical encoding technique such as one-hot encoding, dummy encoding, effect encoding, hash encoding, and the like.

The elements of the trial configuration vector associated with each of the above features may be obtained from a number of different data sources. As shown in FIG. 2, the elements 206-1, 206-2, 206-3 of the trial configuration vector 202 associated with feature “A” are obtained from the first data source 218 whilst the element 210 of the trial configuration vector 202 associated with feature “C” is obtained from the second data source 220. In this example, the first data source 218 may correspond to a pharmacological database or other source which contains information related to the uses, effects, etc. of different drugs. The second data source 220 may correspond to a database or other source related to the historical performance of clinical trial sponsors. Features such as the biological features, chemical features, and sponsor features may be obtained from publicly available databases such as the US and EU clinical trials register, ChemBL, and the like. Some features, such as keyword features, may be extracted from metadata associated with records within such databases (e.g., from web pages associated with a study).

Each element of a trial configuration vector for a clinical trial may be either fixed or optimizable. A fixed element of a trial configuration vector is to be understood as being immutable. That is, during subsequent processing or optimization, a fixed element of a trial configuration vector does not change. An optimizable element of a trial configuration vector is to be understood as being changeable. That is, during subsequent processing or optimization, an optimizable element of a trial configuration may vary or change. In embodiments, the elements of a trial configuration which are fixed or optimizable are predetermined. These elements may be identified by metadata associated with the plurality of features of the clinical trial. Whether an element is fixed or optimizable may be dependent on the feature to which the element relates. For example, features related to the pharmacology of the drug to which the clinical trial is directed may be fixed whilst certain features related to the design and operation of the clinical trial may be optimizable. Beneficially, by dichotomizing the trial configuration vector into fixed and optimizable elements, the configuration of the clinical trial can be processed and/or optimized whilst retaining meaningful outcomes. The identification of fixed and optimizable elements thus helps an updated or optimized clinical trial configuration to have an achievable outcome thus enabling optimization of a clinical trial configuration.

Referring once again to FIG. 1, the trial configuration vector 118 is used by the trial outcome predictor 102 to determine the probabilistic model 120 of the outcome of the clinical.

The trial outcome predictor 102 comprises a machine learning model which may be an unsupervised model, a supervised model, or an ensemble model (i.e., an ensemble of unsupervised and/or supervised models). The machine learning model may be one of a k-nearest neighbor model, a random forest model, an elastic net model, or a support vector machine (SVM) model. The skilled person will appreciate that the present disclosure is not intended to be limited solely to such models, and any suitable machine learning model or predictive model (e.g., rules-based models, fuzzy models, probabilistic models, etc.) may be used.

In one embodiment, the trial outcome predictor 102 comprises an ensemble model which combines predictions from a set of unsupervised and/or supervised models by defining weighting coefficients for each model within the ensemble which minimize cross-validated risk (such as mean squared error). Each model within the ensemble model may comprise one or more hyperparameters. For example, the neighborhood size parameter, k, of a k-nearest neighbor model or the minimum node size parameter of a random forest model. Each model may be associated with a set of possible hyperparameters. The ensemble model may then be learnt by identifying the best performing model (i.e., model+hyperparameter choice) from within these sets.

In one example implementation, the ensemble model comprises a k-nearest neighbor model, a random forest model, an elastic net model, and a support vector machine (SVM) model. The k-nearest neighbor model has a possible parameter set of k=[2,10]. The random forest model has a minimum node size parameter taken from the set {1, 2, 3}, and a second parameter taken from the set

$$⌈ \sqrt{n} \times \{ \frac{1}{2} , 1 , 2 \} ⌉$$

where n corresponds to total number of features within the trial configuration vector. Here, the second parameter corresponds to the number of features to sample (randomly) as candidates at each split. The elastic net model has a λ parameter taken from the set {10, 20, 30, 40, 50, 60, 70, 80, 90, 100} and an α parameter taken from the set 2^{-{5,4,3,2,1}}. The SVM model utilizes an RBF kernel with a cost parameter taken from the set {0.1,1,5,10,50,100,500}. The weights to assign to each model, and the hyperparameter tuning, is performed using 30 repeats of a 5-fold cross validation approach on a training data set (as described in more detail below).

In an embodiment, the trial outcome predictor 102 comprises a causal model. For example, the trial outcome predictor 102 may comprise a Bayesian network or a deconfounder based model. One example of such a model is a probabilistic principal components analysis (PPCA) model fit using stochastic variational inference (SVI) with evidence lower bound (ELBO) optimization. The causal model thus estimates a causal relationship between a trial configuration and an outcome of the clinical trial. The causal model may then be used for causal inference (i.e., determining an outcome for the clinical trial when elements within the clinical trial vector change).

As shown in FIG. 1, the trial outcome predictor 102 may be trained on historical clinical trial data 110 using a training unit 108. Here, the training unit 108 may be understood as a computational unit or unit which trains a machine learning model on training data. The training unit 108 therefore may be separate from the other units of the system 100. For example, the training unit 108 may be a part of an external system specifically configured to utilize specialized hardware and/or software to train the outcome predictor. The training unit 108 may utilize a suitable training algorithm, such as stochastic gradient descent, ADAM, or the like to produce the trained machine learning model. For ensemble models, the training unit 108 may simultaneously train (i.e., fit) each individual model using a suitable training approach and determine the best performing model and a weighted average of all models. The skilled person will appreciate that any suitable training algorithm for the machine learning model used may be utilized by the training unit 108.

In one example implementation, the historical clinical trial data comprises trial configuration data corresponding to 9,297 historical clinical trials conducted prior to 2018. The data includes information relating to 5,409 positive trials (i.e., clinical trials having a successful outcome) and 3,888 negative trials (i.e., clinical trials having an unsuccessful outcome). Each clinical trial within the data is represented by a trial configuration vector having 553 elements with features relating to the biological, chemical, design and operation, keyword, and miscellaneous features described above. The trial outcome predictor 102 may be trained to predict a single outcome for a given trial configuration vector. For example, a first trial outcome predictor may be used to predict the probability of the clinical trial progressing from Phase I to Phase II, whilst a second trial outcome predictor may be used to predict the probability of a severe adverse event occurring. Other possible trial outcomes are described in more detail below.

The trial outcome predictor 102 receives the trial configuration vector 118 as input and provides the probabilistic model 120 of an outcome of the clinical trial as output. The probabilistic model may include a probability score, or probability value, associated with the outcome of the clinical trial. The probability score, or probability value, is representative of a probability that the outcome of the clinical trial will be achieved. The probabilistic model may further comprise an uncertainty estimate. The uncertainty estimate may be associated with the probability score. In embodiments, the probabilistic model is a probability distribution, such as a probability density function, associated with the outcome of the clinical trial. The probability density function may be determined from predictions obtained by the trial outcome predictor 102 using a parametric or non-parametric density estimation approach.

The probabilistic model 120 is representative of a probability of an outcome of the clinical trial being achieved. As such, different trial outcome predictors may be trained and used to provide predictions of different trial outcomes. In one embodiment, the trial outcome corresponds to the overall success of the clinical trial such that the trial outcome predictor is trained to predict a probability score comprising a probability of success of the clinical trial. In one embodiment, the trial outcome corresponds to the clinical trial proceeding from a first stage to a second stage such that the trial outcome predictor is trained to predict a probability score comprising a probability of the clinical trial moving from a first phase to a second phase. Alternatively, the trial outcome corresponds to the clinical trial proceeding from a second stage to a third stage such that the trial outcome predictor is trained to predict a probability score comprising a probability of the clinical trial moving from a second phase to a third phase. In further embodiments, the trial outcome comprises a severe adverse event occurring such that the trial outcome predictor is trained to predict a probability score comprising a probability of a severe adverse event occurring as part of the clinical trial. Examples of severe adverse events include intervention to prevent permanent impairment or damage, disability or permanent damage, hospitalization, and death. When training the different trial outcome predictors described above, the outcomes (e.g., success of a clinical trial, occurrence of a severe adverse event, etc.) are included as targets within the training data.

According to an aspect of the present disclosure, the factors which lead to a predicted trial outcome can be determined to help improve model interpretation and subsequent optimization of a clinical trial configuration. These factors may be represented as contribution scores. As such, the explainability model 104 uses the probabilistic model 120 determined by the trial outcome predictor 102 to determine the plurality of contribution scores 122 for the trial configuration vector 118. As will be described in more detail below, the plurality of contribution scores 122 are indicative of a relative contribution of each element (feature value) in the trial configuration vector 118 to the outcome of the clinical trial.

The explainability model 104 comprises an explainability algorithm which determines the plurality of contribution scores 122. In embodiments, the explainability algorithm utilizes both the probabilistic model 120 and the machine learning model of the trial outcome predictor 102 to determine the plurality of contribution scores 122.

In general, the explainability algorithm used by the explainability model 104 determines the relative contribution, or influence, that each feature of the clinical trial vector 118 makes to the outcome of the clinical trial. The relative contribution can be either positive or negative such that a particular feature value, or element, of the clinical trial vector 118 can either positively or negatively influence the outcome of the clinical trial. In one embodiment, the explainability algorithm determines a relative contribution for a feature using a feature permutation approach. A baseline measurement s_{b} (e.g., a probability associated with an outcome of the clinical trial) is obtained from the trial outcome predictor 102 given the clinical trial vector 118. The element, i, within the clinical trial vector 118 which is associated with the feature is then permuted to generate a transformed clinical trial vector. A permuted measurement, sᵢ, is obtained from the trial outcome predictor 102 given the transformed clinical trial vector. The difference between the baseline measurement and the permuted measurement, i.e., s_{b}−sᵢ, is recorded and the element permutation process is repeated over several iterations to obtain an average of the difference between the two measurements. This average represents the contribution of the element (i.e., feature) to the overall outcome of the clinical trial. A positive average value is indicative of an increase in performance (i.e., an improvement to the trial outcome) when the element is included in the clinical trial vector 118. A negative average value is indicative of a decrease in performance when the element is included in the clinical trial vector 118.

Alternatively, in a further embodiment, when the trial outcome predictor 102 utilizes a random forest model, the explainability algorithm comprises a random forest feature importance algorithm based on the mean decrease in impurity (e.g., decrease in mean squared error, Gini, log loss, etc.). In another embodiment, the explainability algorithm comprises a model agnostic method such as breakDown, LIME, SHAP, or the like.

The explainability algorithm may be applied to all features of the clinical trial vector 118 to obtain a contribution score for each element of the clinical trial vector 118. Alternatively, the explainability algorithm may be applied to a subset of features of the clinical trial vector 118. For example, the explainability algorithm may be applied only to those elements of the clinical trial vector 118 which are optimizable. By focusing on the optimizable elements of the clinical trial vector 118, the plurality of contribution scores 122 provide a compact representation of the impact of the features of the clinical trial that are changeable thus providing insight into which features may be selected for further processing or optimization. In this way, the explainability model of the present disclosure provides a view into the model (e.g., the trial outcome predictor) and its related output by providing a data-based and/or visual representation or otherwise explanations of how the training data impacts or otherwise determines the output of the disclosed AI model, e.g., the trial outcome predictor. Said another way, the explainability model allows for a window or view into how the AI model is currently trained and how such training impacts the output result. This allows for the AI-model to be retrained or reconfigured, e.g., with different training data, such as different trial configuration vectors having different fixed and/or optimizable parameters and/or with different data from additional data sources, in order to eliminate error and/or bias in a second version of an AI model, e.g., trial outcome predictor and its related output.

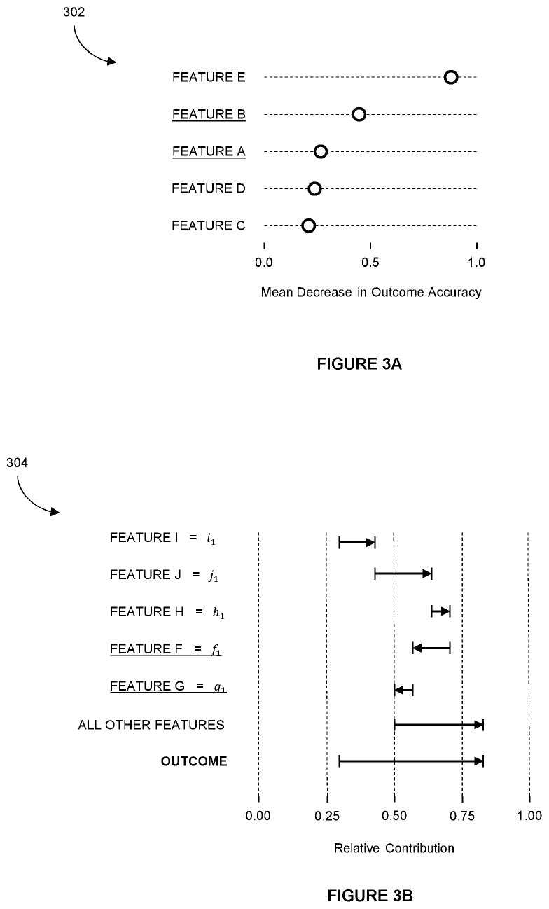

FIGS. 3A and 3B show example contribution scores according to embodiments of the present disclosure.

FIG. 3A shows a plurality of contribution scores 302 (e.g., the plurality of contribution scores 122 shown in FIG. 1) for five different features “A”-“E”. Features “C”, “D”, and “E” are fixed features (i.e., these features have fixed elements within the clinical trial vector) whilst features “A” and “B” are optimizable features (i.e., these features have adjustable elements within the clinical trial vector) as indicated by the underlined text. In the example shown in FIG. 3A, the plurality of contribution scores 302 are determined using an explainability model for a random forest based trial outcome prediction model such that the plurality of contribution scores 302 illustrate the mean decrease in accuracy outcome for each feature. This metric may be understood as being the loss in accuracy (i.e., when predicting the outcome of the clinical trial) which would occur if a corresponding feature were to be removed from the clinical trial vector. The plurality of contribution scores 302 thus encodes the relative importance of each feature to the overall outcome of the clinical trial. In the example shown in FIG. 3A, the features are ordered such that removal of feature “E” would lead to the greatest decrease in outcome accuracy thus indicating that feature “E” is the most important feature to the overall outcome of the clinical trial.

FIG. 3B shows a plurality of contribution scores 304 (e.g., the plurality of contribution scores 122 in FIG. 1) for five different features “F”-“J”. Features “H”, “I”, and “J” are fixed features, whilst features “F” and “G” are optimizable features. The plurality of contribution scores 304 further includes the contribution score for all other features within the clinical trial vector. FIG. 3B also shows the overall outcome determined from the probabilistic model of the clinical trial. The plurality of contribution scores 304 shown in FIG. 3B are determined using breakDown, a model agnostic explainability model, and correspond to the contribution that each of features “F”-“J” make to the overall outcome when said features take a particular value. That is, the contribution score for a feature corresponds to the contribution that the feature makes to the overall outcome given the value, or element, of that feature within the clinical trial vector. In the example shown in FIG. 3B, feature “I” having an element i₁ in the clinical trial vector results in an increase in the outcome. This increase is indicated by the left-right arrow which indicates the difference in outcome without feature “I” (left hand side of arrow) and with feature “I” (right hand side of arrow). In contrast, feature “F” having an element f₁ in the clinical trial vector results in a decrease in the outcome. This decrease is indicated by the right-left arrow which indicates the difference in outcome with feature “F” (left hand side of arrow) and without feature “F” (right hand side of arrow).

The contribution scores shown in FIGS. 3A and 3B provide insight into the impact that the elements of the clinical trial vector have on the predicted trial outcome. This insight may help drive improvement to the clinical trial which may subsequently improve the overall likelihood of the trial outcome being achieved. In addition, distinguishing between the contributions provided by the fixed and optimizable parameters may help drive the optimization process by identifying elements which may provide the greatest improvement to the trial outcome when optimized.

Referring once again to FIG. 1, the probabilistic model 120 and one or more of the plurality of contribution scores 122 are used to form an explainable prediction 124 of the trial outcome of the clinical trial. The one or more of the plurality of contribution scores 122 used to generate the explainable prediction 124 correspond to the contribution scores associated with the optimizable elements of the clinical trial vector 118. Consequently, the explainable prediction 124 is indicative of what improvements may be made to increase the likelihood of the trial outcome being achieved.

The explainable prediction 124 is output for review by the user 116. The explainable prediction 124 may be output in a form similar to that described in relation to FIGS. 3A and 3B above. Alternatively, the explainable prediction 124 may be output in structured form (e.g., in a JSON file) for further processing or handling. In one embodiment, the explainable prediction 124 is included in the report 114 which is output for review by the user 116.

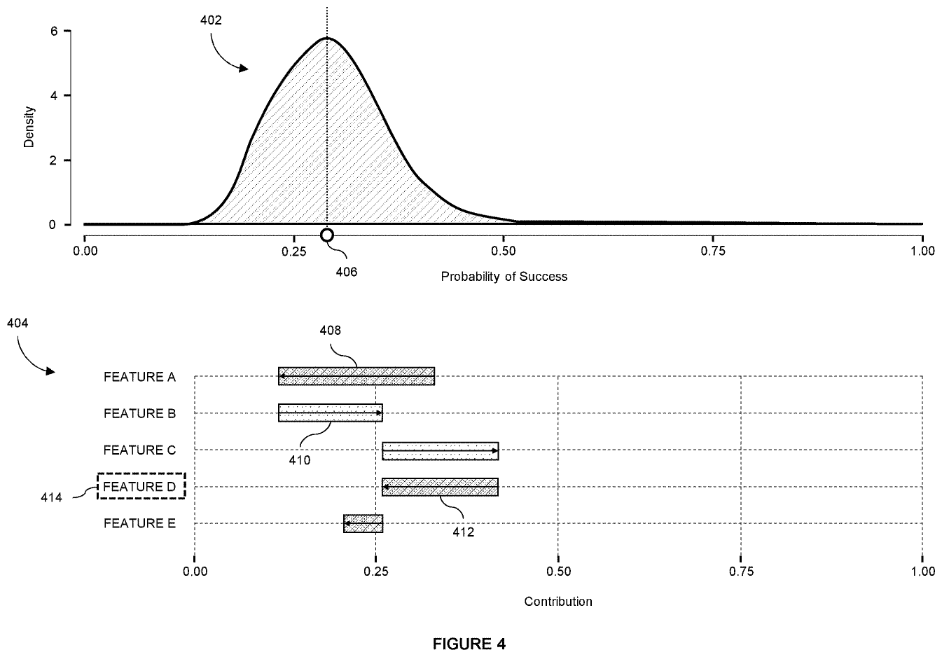

FIG. 4 shows a portion of an example report according to embodiments of the present disclosure.

FIG. 4 shows a probabilistic model 402 of an outcome of a clinical trial and a plurality of contribution scores 404. An overall probability of success 406 is shown alongside the probabilistic model 402. The plurality of contribution scores 404 include a first contribution score 408, a second contribution score 410, and a third contribution score 412. The third contribution score 412 is associated with an optimizable feature 414.

In the example of FIG. 4, the clinical trial corresponds to a phase II study of two interventions in patients with advanced urothelial carcinoma. The outcome corresponds to the overall success of the clinical trial such that the probabilistic model 402 comprises a posterior probability distribution of the probability of success of the clinical trial. The probabilistic model 402 may be determined using a trial outcome predictor such as the trial outcome predictor 102 of the system 100 of FIG. 1. The overall probability of success 406 is approximately 0.3 with uncertainty estimates (95% confidence intervals) of 0.18 and 0.44. The plurality of contribution scores 404 may be determined using an explainability model such as the explainability model 104 of the system 100 of FIG. 1. The plurality of contribution scores 404 are ordered according to size of their contribution to the overall probability of success 406 of the clinical trial. The skilled person will appreciate that the labeling of the features (e.g., “A”, “B”, etc.) in the plurality of contribution scores 404 is done for illustrative purposes. The first contribution score 408 is associated with feature “A” which corresponds to a design and operation feature of the clinical trial. Particularly, feature “A” corresponds to the number of terminated trials associated with a sponsor of the clinical trial. The first contribution score 408 has an overall negative contribution to the predicted outcome of the clinical trial and is therefore responsible for a decrease in the overall probability of success. The first contribution score 408 thus indicates that the biggest single factor contributing to the overall probability of success 406 is the number of trials associated with one of the sponsors of the clinical trial that have been terminated. The second contribution score 410 is associated with feature “B” which corresponds to another design and operation feature of the clinical trial. Particularly, feature “B” corresponds to the number of sponsors involved in the clinical trial. The second contribution score 410 has an overall positive contribution and is thus responsible for an increase in the overall probability of success. The third contribution score 412 is associated with feature “D” which corresponds to another design and operation feature of the clinical trial. Particularly, feature “D” corresponds to the number of investigators involved in the clinical trial. The third contribution score 412 has an overall negative contribution and is thus responsible for a decrease in the overall probability of success. In this instance, feature “D” is an optimizable feature which means that the element in the clinical trial vector associated with feature “D” is modifiable. This indicates that adjusting the number of investigators involved in the clinical trial may help to improve the overall probability of success. The plurality of contribution scores 404 included in the example report shown in FIG. 4 thus help to identify potential improvements to the clinical trial. These improvements may optimize the probability of the outcome of the clinical trial being achieved which may thus improve the overall design of the clinical trial.

As such, the information included in a report may be used to update the configuration of the clinical trial (i.e., update one or more of the optimizable elements of the clinical trial vector). In one embodiment, the updated configuration of the clinical trial is obtained from an external source such as a user or an external system. Alternatively, the updated configuration of the clinical trial is obtained by an optimization process.

Referring once again to FIG. 1, the first updated trial configuration vector 126 may be obtained from an external source (e.g., the user 116) to determine an updated probabilistic model of the outcome of the clinical trial from the trial outcome predictor 102 based on the first updated trial configuration vector 126 (in the same manner as described above in relation to the trial configuration vector 118). The first updated trial configuration vector 126 comprises one or more optimized elements based on the explainable prediction 124. Here, an optimized element within the first updated trial configuration vector 126 corresponds to an update, change, or adjustment, to an optimizable element within the trial configuration vector 118. As such, the first updated trial configuration vector 126 corresponds to the trial configuration vector 118 with one or more elements which have been adjusted or optimized by an external source (e.g., the user 116 or another system). The updated probabilistic model may then be output for review by the user 116. In one embodiment, a plurality of updated contribution scores for the updated trial configuration are determined using the explainability model 104 (in the same manner as described above in relation to the plurality of contribution scores 122). The plurality of updated contribution scores may then be compared to the plurality of contribution scores 122 to determine any changes to the contribution scores in consequence of updating the one or more optimizable elements of the trial configuration vector 118. The result of the comparison may also be output for review by the user 116.

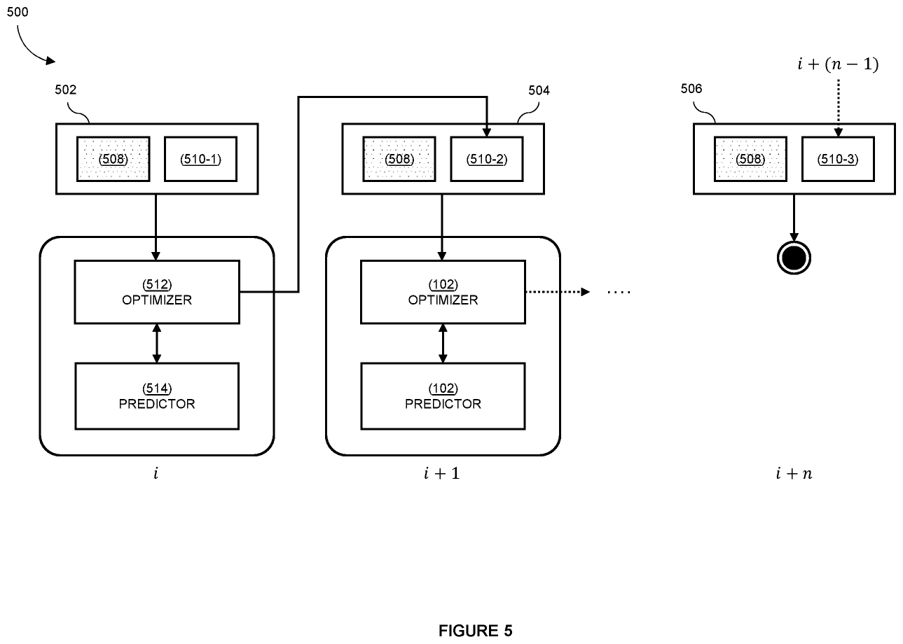

In an alternative embodiment, the trial configuration vector 118 may be updated using an optimization process employed by the optimizer 106. An example optimization process is illustrated in FIG. 5.

FIG. 5 shows an example optimization process 500 according to embodiments of the present disclosure.

FIG. 5 shows a first trial configuration 502 (trial configuration vector) associated with a clinical trial, a first updated trial configuration 504, and a second updated trial configuration 506. The first trial configuration 502 comprises fixed trial parameter values 508 and an optimizable trial parameter value 510-1. The first updated trial configuration 504 comprises the fixed trial parameter values 508 and a first updated optimizable trial parameter value 510-2. The second updated trial configuration 506 comprises the fixed trial parameter values 508 and a second updated optimizable trial parameter value 510-3. FIG. 5 further shows an optimizer 512 which may work in conjunction with a predictor 514 to determine an updated trial configuration. In one embodiment, the optimizer 512 corresponds to the optimizer 106 of the system 100 of FIG. 1 and the predictor 514 corresponds to the trial outcome predictor 102 of the system 100 of FIG. 1.

The example optimization process 500 comprises steps, i, i+1, . . . , i+n. At the first step, i, the first trial configuration 502 associated with the clinical trial is optimized to create the first updated trial configuration 504. The optimization performed at step i results in an improvement to an outcome of the clinical trial (e.g., the optimization results in the probability of the clinical trial moving from phase I to phase II being increased). The first updated trial configuration 504 created at the first step, i, comprises the same values, or elements, as the first trial configuration 502 for the fixed trial parameter values 508 but with the first updated optimizable trial parameter value 510-2 determined by the optimizer 512. At the next step, i+1, this process is repeated, but now using the first updated trial configuration 504, to determine a new trial configuration which improves the outcome of the clinical trial. The process is repeated until the final step, i+n, where the process terminates. As such, the second updated trial configuration 506, determined at step i+(n−1), is output from the optimization process 500 as the final, or optimized, trial configuration.

In one embodiment, the optimizer 512 obtains or generates an updated trial configuration using a greedy heuristic. At a general level, such an approach evaluates, at each step, the performance of several candidate trial configurations and chooses the best performing candidate trial configuration as the updated trial configuration. Here, performance may be measured using the predictor 514 and thus corresponds to the estimated outcome (e.g., probability of success) of the clinical trial given a trial configuration. The candidate trial configurations may be determined by obtaining or generating configurations within the neighborhood of the current trial configuration (e.g., by permuting the optimizable trial parameter value or values). The candidate trial configuration which provides the greatest improvement to the estimated outcome is then selected as the best performing candidate trial configuration. In other embodiments, the optimizer 512 uses an optimization algorithm such as hill climbing, tabu search, simulated annealing, or the like to obtain or generate the updated trial configuration. The skilled person will appreciate that the present disclosure is not intended to be limited to such optimization approaches, and any suitable algorithm or method may be used to obtain an optimized trial configuration which provides an improved outcome of the clinical trial.

In one embodiment, the optimizable trial parameter value 510-1 to which the optimization process 500 is applied is selected based on a contribution score associated with that value. For example, contribution scores may be obtained or generated for the optimizable elements of a trial configuration vector (such as those within the plurality of contribution scores 122 shown in FIG. 1). If an optimizable element has a contribution score which meets a predetermined criteria, then the optimizable element is selected for optimization. Examples of predetermined criteria include the contribution being negative, the contribution score being below a predetermined threshold, and the contribution score being associated with a certain feature. An optimization process (such as the optimization process 500) is used to optimize the optimizable element such that the overall outcome of the clinical trial is improved. In this way, the system is automatically able to identify aspects of the clinical trial which may be improved and optimize these elements to improve the likelihood of the trial outcome for the clinical trial being met. This provides an efficient and effective mechanism for improving the design of a clinical trial and helps improve the likelihood of the clinical trial achieving a trial outcome before the clinical trial is started.

The final trial configuration, i.e., the second updated trial configuration 506 in FIG. 5, is obtained once the optimization approach used by optimizer 512 has terminated. The optimization approach may terminate once a predetermined number of steps have been performed. Alternatively, the optimization approach may terminate once the improvement to the outcome of the clinical trial achieved by subsequent iterations is less than a predetermined amount or has not changed over a set number of iterations.

The system described in relation to FIGS. 1 to 5 above may be used to provide an efficient and accurate prediction of an outcome of a clinical trial. By providing an explainable prediction of the outcome, the contribution of the different features of the clinical trial can be reviewed thereby enabling greater insight into the prediction. Moreover, the explainable prediction may help drive the optimization of the clinical trial by identifying the features of the clinical trial which may be optimized to help improve the likelihood of the trial outcome being achieved.

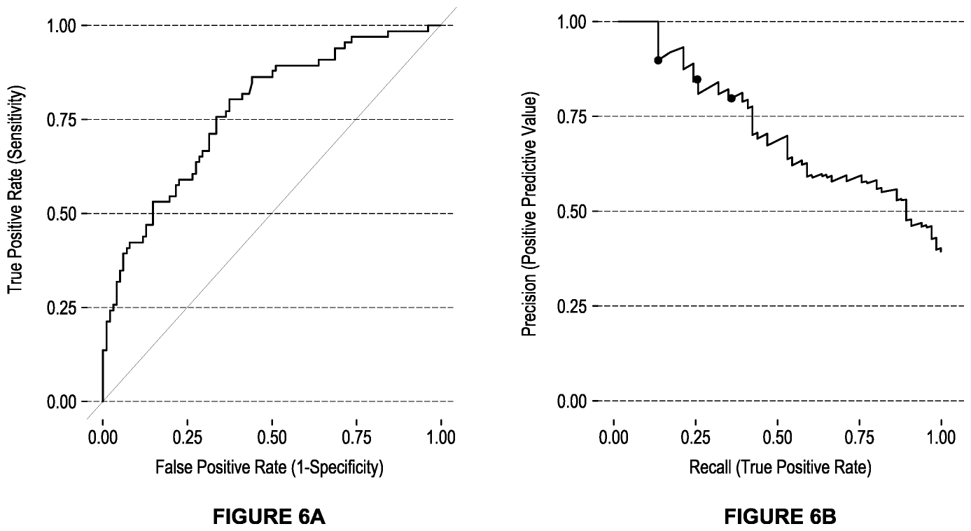

FIGS. 6A and 6B show the results of the system of the present disclosure applied to predicting the outcome of a plurality of clinical trials.

The results shown in FIGS. 6A and 6B correspond to the results of using the approach described in relation to FIGS. 1-5 to predict the outcome (positive outcome or negative outcome) for Phase III oncology trials. An elastic net model was used for the trial outcome predictor and was trained on 1,982 trials completed before 1 Jan. 2018 and the results shown in FIGS. 6A and 6B were obtained from a held back test set of 168 trials completed after 1 Jan. 2018. The training data comprised 779 successful trials and 1,203 failed trials. The test data comprised 66 successful trials and 102 failed trials. Features for each trial included the sponsor type (e.g., government, pharma, etc.), the target type, the mechanism of action, MESH terms associated with the trial, the trial region, and the trial country.

FIG. 6A shows a receiver operating characteristic (ROC) curve of the true positive rate (sensitivity) and false positive rate (1−specificity) for the results obtained on the test set. FIG. 6B shows the precision recall graph for the results obtained on the test set. The system achieved an AUC of 0.773, an AUPR of 0.699, and an approximately 85% precision and 10% recall.

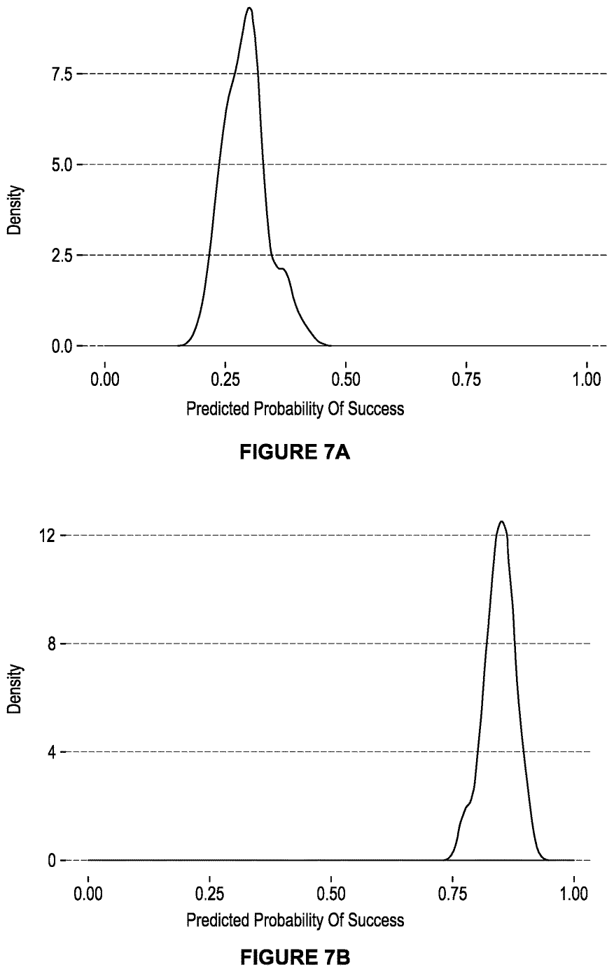

FIGS. 7A and 7B show the predicted probability of success for two clinical trials according to embodiments of the present disclosure.

FIG. 7A shows the probability of success obtained by the system 100 of FIG. 1 for a first clinical trial of axitinib for renal cell carcinoma (RCC). FIG. 7B shows the probability of success obtained by the system 100 of FIG. 1 for a second clinical trial of axitinib for RCC. As shown, the first clinical trial has a predicted probability of success of 0.29 (±0.05) and the second clinical trial has a predicted probability of success of 0.84 (±0.03). Of note is that both the first clinical trial and the second clinical trial had the same sponsor, the same indication, and the same drug. However, by incorporating richer features from across different categories into the clinical trial vector (e.g., biological features, chemical features, design and operation features, etc.), the approach of the present disclosure was able to predict correctly that the first clinical trial was likely to fail whilst the first clinical trial was likely to succeed.

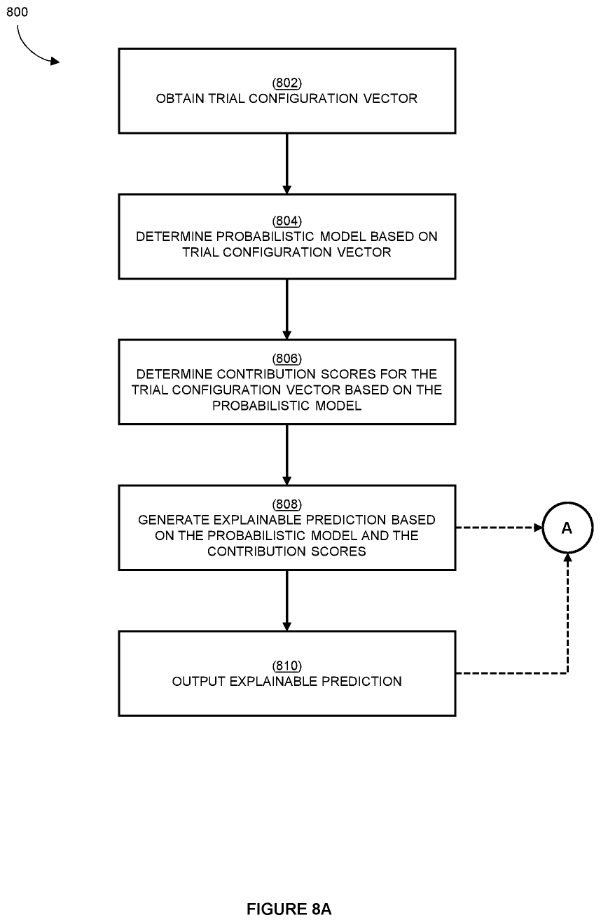

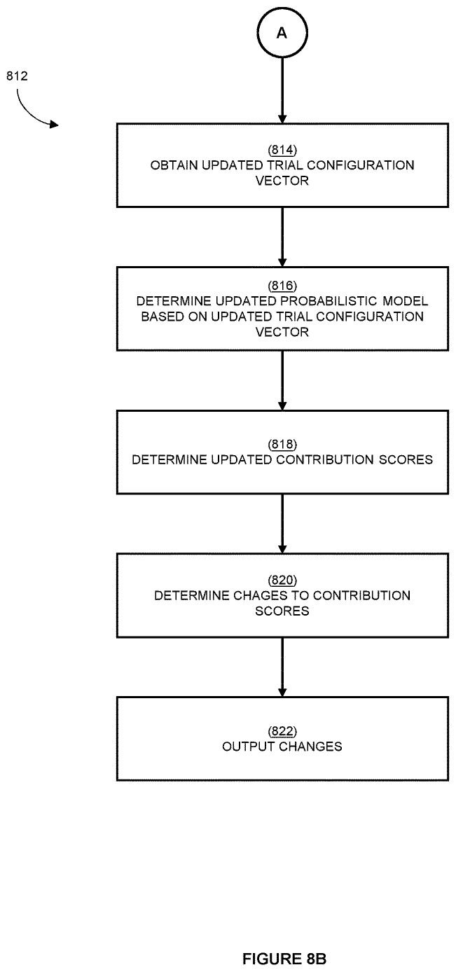

FIG. 8A shows a method 800 for generating an explainable prediction of a trial outcome of a clinical trial according to embodiments of the present disclosure.

The method 800 comprises the steps of obtaining 802 a trial configuration vector, determining 804 a probabilistic model based on the trial configuration vector, determining 806 contribution scores for the trial configuration vector based on the probabilistic model, generating 808 an explainable prediction of the trial outcome based on the contribution scores and the probabilistic model, and outputting 810 the explainable prediction.

At the step of obtaining 802, a trial configuration vector (e.g., trial configuration vector 118 of the system 100 in FIG. 1) associated with the clinical trial is obtained from one or more data sources (e.g., one or more data sources 112 of the system 100 in FIG. 1). Additionally, or alternatively, obtaining the trial configuration vector may comprise generating the trial configuration vector from the one or more data sources. In such aspects, generation may comprise altering elements (e.g., to be fixed and/or optimized) of the trial configuration (updated other otherwise) to determine, select, or create a trial and/or trial configuration vector. The trial configuration vector encodes aspects related to the design and protocol of the clinical trial and comprises one or more fixed elements and one or more optimizable elements. Each element, or value, of the trial configuration vector is associated with a feature of the clinical trial and may be binary valued, integer valued, or real valued. The clinical trial to which the trial configuration vector relates may be a proposed clinical trial which has not yet begun, or an active clinical trial which has already begun.

The trial configuration vector may comprise one or more elements associated with one or more biological features, wherein the one or more biological features are related to a target associated with the clinical trial. The one or more biological features may comprise at least one hierarchical mechanism of action feature. The trial configuration vector may comprise one or more elements associated with one or more chemical features, wherein the one or more chemical features are related to a target associated with the clinical trial. The trial configuration vector may comprise one or more elements associated with one or more design and operation features of the clinical trial. The one or more design and operation features may include one or more geographical features related to a site associated with the clinical trial. The one or more design and operation features may include one or more sponsor features related to a sponsor associated with the clinical trial. The one or more design and operation features may include one or more investigator features related to an investigator associated with the clinical trial. The trial configuration vector may comprise one or more elements associated with keywords associated with the clinical trial. The trial configuration vector may comprise miscellaneous features related to various aspects of the clinical trial not covered by the above feature groupings.

At the step of determining 804, a probabilistic model (e.g., probabilistic model 120 of the system 100 in FIG. 1) of an outcome of the clinical trial is determined using a trial outcome predictor (e.g., trial outcome predictor 102 of the system 100 in FIG. 1) based on the trial configuration vector. In one embodiment, the outcome corresponds to the overall success of the clinical trial such that the trial outcome predictor predicts a probability score comprising a probability of success of the clinical trial. In one embodiment, the outcome corresponds to the clinical trial proceeding from a first stage to a second stage such that the trial outcome predictor predicts a probability score comprising a probability of the clinical trial moving from a first phase to a second phase. Alternatively, the outcome corresponds to the clinical trial proceeding from a second stage to a third stage such that the trial outcome predictor predicts a probability score comprising a probability of the clinical trial moving from a second phase to a third phase. In further embodiments, the outcome comprises a severe adverse event occurring such that the trial outcome predictor predicts a probability score comprising a probability of a severe adverse event (e.g., intervention to prevent permanent impairment or damage, disability or permanent damage, hospitalization, death, and the like) occurring as part of the clinical trial.

The trial outcome predictor comprises a prediction model which has been trained on data related to a plurality of historical clinical trials. Further details regarding training a trial outcome predictor is given above in relation to the training unit 108 of the system 100 of FIG. 1. The trial outcome predictor comprises a machine learning model which may be an unsupervised model or a supervised model. Examples of such models include a k-nearest neighbor model, a random forest model, an elastic net model, and a support vector machine. Alternatively, the trial outcome predictor may comprise an ensemble model.

The probabilistic model of the outcome of the clinical trial comprises a probability score associated with the outcome of the clinical trial. The probability score, or probability value, is representative of a probability that the outcome of the clinical trial will be achieved. The probability score may comprise a probability of the clinical trial moving from a first phase to a second phase. The probability score may comprise a probability of the clinical trial moving from a second phase to a third phase. The probability score may comprise a probability of a severe adverse event occurring as part of the clinical trial. The probabilistic model of the outcome of the clinical trial may further comprise an uncertainty estimate.

At the step of determining 806, a plurality of contribution scores (e.g., plurality of contribution scores 122 of the system 100 of FIG. 1) for the trial configuration vector are determined using an explainability model (e.g., explainability model 104 of the system 100 of FIG. 1) based on the probabilistic model. Each contribution score of the plurality of contribution scores is indicative of a relative contribution of an associated element of the trial configuration vector to the outcome of the clinical trial. Example contribution scores are shown and described in relation to FIGS. 3A and 3B above.

At the step of generating 808, an explainable prediction (e.g., explainable prediction 124 of FIG. 1) of the trial outcome of the clinical trial is generated based on the probabilistic model and one or more contribution scores of the plurality of contribution scores. The one or more contribution scores being associated with the one or more optimizable elements. In some embodiments, the explainable prediction further comprises one or more further contribution scores associated with the one or more fixed elements.

At the step of outputting 810, the explainable prediction is output for review by a user (e.g., user 116 shown in FIG. 1). In some embodiments, the explainable prediction is included in a report (e.g., report 114 shown in FIG. 1) such that the report is output for review by a user. A portion of an example report is illustrated and described in relation to FIG. 4 above.

FIG. 8B shows a method 812 comprising further steps which may be performed as part of the method 800 of FIG. 8A according to embodiments of the present disclosure.

The steps of the method 812 may be performed after the steps of method 800 have been completed. Particularly, the method 812 may be performed after the step of generating 808 an explainable prediction or the step of outputting 810 the explainable prediction.

The method 812 comprises the steps of obtaining 814 an updated trial configuration vector, determining 814 an updated probabilistic model based on the updated trial configuration vector, determining 818 updated contribution scores based on the updated probabilistic model, determining 820 changes to the contribution scores, and outputting 822 the changes to the contribution scores.

At the step of obtaining 814, an updated trial configuration vector (e.g., first updated trial configuration vector 126 or second updated trial configuration vector 126 shown in FIG. 1) associated with the clinical trial is obtained. The updated trial configuration vector comprises one or more optimized elements based on the explainable prediction. The updated trial configuration vector may be obtained from an external source such as the user or an external computer system. Additionally, or alternatively, obtaining a first trial configuration (updated or otherwise) may comprise generating the first trial configuration from one or more data sources. In such aspects, generation may comprise altering the elements (e.g., to be fixed and/or optimized) of the trial configuration (updated other otherwise) to determine, select, or create a trial and/or trial configuration vector.

At the step of determining 816, an updated probabilistic model of the outcome of the clinical trial is determined using the trial outcome predictor based on the updated trial configuration vector.

Optionally, after the updated probabilistic model has been determined, the method 812 outputs the updated probabilistic model of the outcome of the clinical trial for review by the user. The updated probabilistic model provides feedback to the user pertaining to the change to the trial outcome occurring as a result of the changes made to the trial configuration vector. This feedback may aid in the explainability and/or optimization of the clinical trial.

At the step of determining 818, a plurality of updated contribution scores for the updated trial configuration vector are determined using the explainability model based on the updated probabilistic model.

At the step of determining 820, one or more changes to the plurality of contribution scores are determined based on a comparison of the plurality of contribution scores and the plurality of updated contribution scores.

At the step of outputting 822, the one or more changes to the plurality of contribution scores are output for review by a user. The user may then review the changes to the trial outcome, and the contribution of each feature to the trial outcome, which occurred as a result of changing the trial configuration vector. This feedback provides a detailed level of insight into the design, operation, and optimization of the clinical which may help to improve the design and execution of clinical trials.

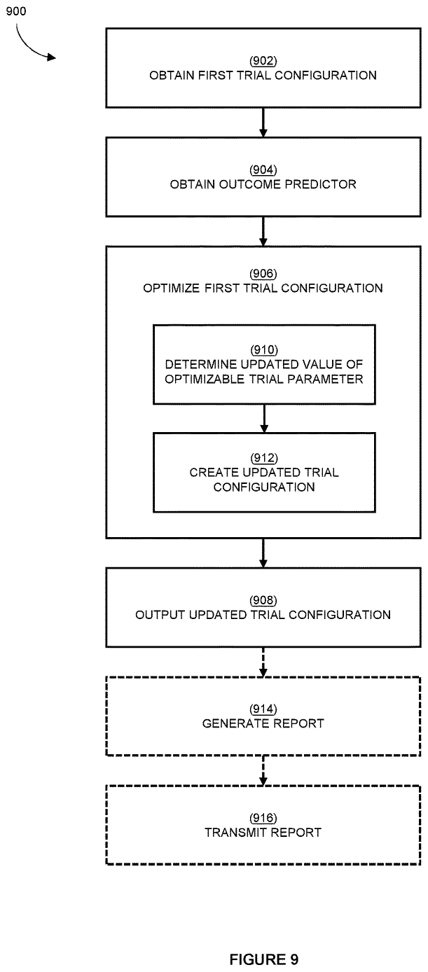

FIG. 9 shows a method 900 for optimizing the parameters of a clinical trial according to embodiments of the present disclosure.

The method 900 comprises the steps of obtaining 902 a first trial configuration associated with a clinical trial, obtaining 904 an outcome predictor, optimizing 906 the first trial configuration to improve an outcome of the clinical trial, and outputting 908 the updated trial configuration. The step of optimizing 906 comprises the steps of determining 910 an updated value of an optimizable trial parameter of the first trial configuration and creating 912 an updated trial configuration including the updated value of the optimizable trial parameter. In some embodiments, the method 900 further comprises the steps of generating 914 a report and transmitting 916 the report.

At the step of obtaining 902, a first trial configuration (e.g., trial configuration vector 118 shown in FIG. 1) associated with a clinical trial is obtained from one or more data sources (e.g., one or more data sources 112 shown in FIG. 1). The first trial configuration comprises values associated with one or more fixed trial parameters and at least one optimizable trial parameter.

At the step of obtaining 904, an outcome predictor (e.g., trial outcome predictor 102 of the system 100 in FIG. 1) is obtained. The outcome predictor estimates a relationship between a trial configuration of a clinical trial and an outcome of the clinical trial.

As described above, the outcome predictor may comprise a supervised model, an unsupervised model, or an ensemble model. In one embodiment, the outcome predictor comprises a causal model. Consequently, the relationship determined by the outcome predictor between the trial configuration of the clinical trial and the outcome of the clinical trial comprises a causal relationship determined by the causal model.

At the step of optimizing 906, the first trial configuration is optimized (e.g., by the optimizer 106 of the system 100 shown in FIG. 1) to improve an outcome of the clinical trial. In this way, the system is automatically able to identify aspects of the clinical trial which may be improved and optimize these elements to improve the likelihood of the trial outcome for the clinical trial being met. This provides an efficient and effective mechanism for improving the design of a clinical trial and helps improve the likelihood of the clinical trial achieving a trial outcome before the clinical trial is started. In one embodiment, an optimizable element within the trial configuration is identified for optimization. The optimizable element may be manually identified (e.g., by a user) or automatically identified based on a contribution score associated with the optimizable element. For example, the identified optimizable element may correspond to the optimizable element within the clinical trial vector which makes the greatest negative contribution to the overall outcome of the clinical trial. In such embodiments, an explainability mod (e.g., the explainability model 104 of the system 100 of FIG. 1) may be used to determine contribution scores for the optimizable elements of the clinical trial vector prior to the step of optimizing 906.

At the step of determining 910, an updated value of the at least one optimizable trial parameter is determined using the outcome predictor and the first trial configuration such that a first estimated outcome of the clinical trial is greater than a second estimated outcome of the clinical trial. The first estimated outcome is determined from the outcome predictor based on the updated trial configuration and the second estimated outcome is determined from the outcome predictor based on the first trial configuration. In one embodiment, the updated value of the at least one optimizable trial parameter is determined using a greedy heuristic as described above. Alternatively, an optimization algorithm such as hill climbing, tabu search, simulated annealing, or the like is used to obtain the updated value of the at least one trial parameter.

At the step of creating 912, an updated trial configuration (e.g., second updated trial configuration vector 128 shown in FIG. 1) comprising the updated value of the at least one optimizable trial parameter is created.

At the step of outputting 908, the updated trial configuration is output for review by a user (e.g., user 116 shown in FIG. 1). Optionally, an estimated outcome associated with the updated trial configuration is also output for review by the user.

In embodiments, the method 900 further comprises the step of generating 914 a report comprising one or more of the values of the updated trial configuration. A portion of an example report is shown and described in relation to FIG. 4 above. The method 900 may further comprise transmitting 916 the report for display to a user. For example, the report may be generated on a first device or system and transmitted (e.g., over a local area network, wide area network, the Internet, or the like) to a second device or system where the report is made available for display to the user. In such a configuration, the system and data used to predict the outcome of the clinical trial and generate the report may be kept separate and secure from the user thereby reducing the user's access to potentially sensitive data used to generate the report.

In some embodiments, the method 900 further comprises determining, using an explainability model (e.g., explainability model 104 of the system 100 shown in FIG. 1), a first plurality of contribution scores for the updated trial configuration. Each contribution score of the first plurality of contribution scores being indicative of a relative contribution of an associated value of the updated trial configuration to the first estimated outcome. In some embodiments, the report further comprises one or more of the first plurality of contribution scores for the updated trial configuration.

The method 900 may further comprise determining, using the explainability model, a second plurality of contributions scores for the first trial configuration. Each contribution score of the first plurality of contribution scores being indicative of a relative contribution of an associated value of the first trial configuration to the second estimated outcome. In some embodiments, the report further comprises one or more of the second plurality of contribution scores for the first trial configuration. In further embodiments, the report further comprises a comparison of the first plurality of contribution scores for the updated trial configuration and the second plurality of contribution scores for the first trial configuration.

The optimization process of FIG. 9 provides an efficient and effective mechanism for improving the design and understanding of a clinical trial. The optimization process further helps improve the probability of a clinical trial achieving a trial outcome before the clinical trial is started.

The systems and methods of the present disclosure (described in relation to FIGS. 1 to 9 above) may be implemented in hardware or a combination of hardware and software. For example, they may be implemented as a dedicated hardware device, a software library, or a network package bound into network applications. In an embodiment, the present disclosure is implemented in software such as a program running on an operating system.

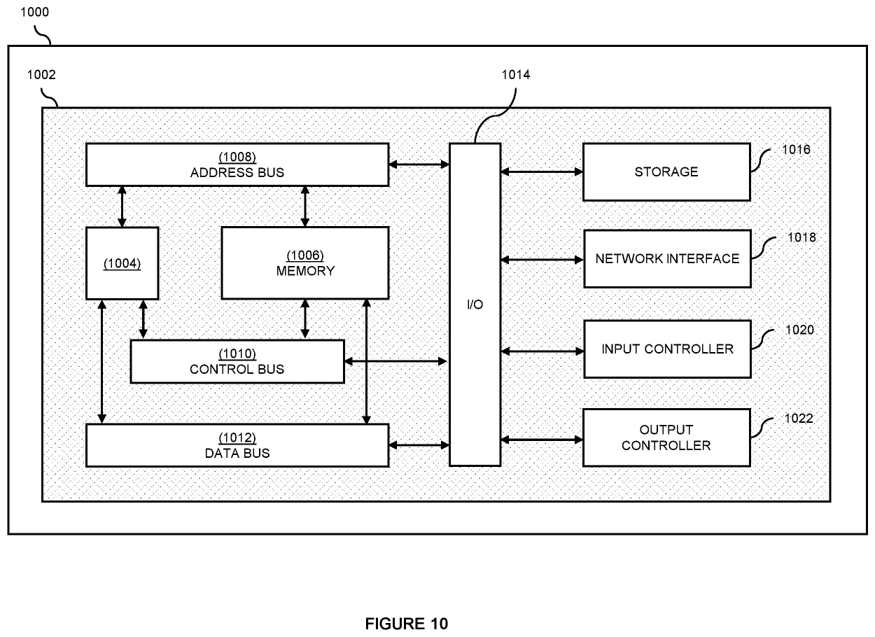

FIG. 10 shows an example computing system for carrying out the methods of the present disclosure. Specifically, FIG. 10 shows a block diagram of an embodiment of a computing system according to example aspects and embodiments of the present disclosure.

Computing system 1000 can be configured to perform any of the operations disclosed herein such as, for example, any of the operations discussed with reference to FIGS. 1 to 10. Computing system includes one or more computing device(s) 1002. One or more computing device(s) 1002 of computing system 1000 comprise one or more processors 1004 and memory 1006. One or more processors 1004 can be any general-purpose processor(s) configured to execute a set of instructions. For example, one or more processors 1004 can be one or more general-purpose processors, one or more field programmable gate array (FPGA), and/or one or more application specific integrated circuits (ASIC). In one embodiment, one or more processors 1004 include one processor. Alternatively, one or more processors 1004 include a plurality of processors that are operatively connected. One or more processors 1004 are communicatively coupled to memory 1006 via address bus 1008, control bus 1010, and data bus 1012. Memory 1006 can be a random-access memory (RAM), a read-only memory (ROM), a persistent storage device such as a hard drive, an erasable programmable read-only memory (EPROM), and/or the like. One or more computing device(s) 1002 further comprise input/output (I/O) interface 1014 communicatively coupled to address bus 1008, control bus 1010, and data bus 1012.

Memory 1006 can store information that can be accessed by one or more processors 1004. For instance, memory 1006 (e.g. one or more non-transitory computer-readable storage mediums, memory devices) can include computer-readable instructions (not shown) that can be executed by one or more processors 1004. The computer-readable instructions can be software written in any suitable programming language or can be implemented in hardware. Additionally, or alternatively, the computer-readable instructions can be executed in logically and/or virtually separate threads on one or more processors 1004. For example, memory 1006 can store instructions (not shown) that when executed by one or more processors 1004 cause one or more processors 1004 to perform operations such as any of the operations and functions for which computing system 1000 is configured, as described herein. In addition, or alternatively, memory 1006 can store data (not shown) that can be obtained, received, accessed, written, manipulated, created, and/or stored. In some implementations, one or more computing device(s) 1002 can obtain from and/or store data in one or more memory device(s) that are remote from the computing system 1000.

Computing system 1000 further comprises storage unit 1016, network interface 1018, input controller 1020, and output controller 1022. Storage unit 1016, network interface 1018, input controller 1020, and output controller 1022 are communicatively coupled via I/O interface 1014.

Storage unit 1016 is a computer readable medium, optionally a non-transitory computer readable medium, comprising one or more programs, the one or more programs comprising instructions which when executed by one or more processors 1004 cause computing system 1000 to perform the method steps of the present disclosure. Alternatively, storage unit 1016 is a transitory computer readable medium. Storage unit 1016 can be a persistent storage device such as a hard drive, a cloud storage device, or any other appropriate storage device.

Network interface 1018 can be a Wi-Fi module, a network interface card, a Bluetooth module, and/or any other suitable wired or wireless communication device. In an embodiment, network interface 1018 is configured to connect to a network such as a local area network (LAN), or a wide area network (WAN), the Internet, or an intranet.

FIG. 10 illustrates one example computing system 1000 that can be used to implement the present disclosure. Other computing systems can be used as well. Computing tasks discussed herein as being performed at and/or by one or more functional unit(s) can instead be performed remote from the respective system, or vice versa. Such configurations can be implemented without deviating from the scope of the present disclosure. The use of computer-based systems allows for a great variety of possible configurations, combinations, and divisions of tasks and functionality between and among components. Computer-implemented operations can be performed on a single component or across multiple components. Computer-implemented tasks and/or operations can be performed sequentially or in parallel. Data and instructions can be stored in a single memory device or across multiple memory devices.

What is claimed is:

1. A computer-implemented method for generating an explainable prediction of a trial outcome of a clinical trial, the computer-implemented method comprising:
obtaining, by one or more processors, from one or more data sources in communication with the one or more processors, a trial configuration vector associated with the clinical trial, the trial configuration vector comprising one or more fixed elements and one or more optimizable elements;
determining, by the one or more processors, using a trial outcome predictor, a probabilistic model of an outcome of the clinical trial based on the trial configuration vector, the trial outcome predictor having been trained on data related to a plurality of historical clinical trials;
determining, by the one or more processors, using an explainability model, a plurality of contribution scores for the trial configuration vector based on the probabilistic model, each contribution score of the plurality of contribution scores being indicative of a relative contribution of an associated element of the trial configuration vector to the outcome of the clinical trial;
generating, by the one or more processors, an explainable prediction of the trial outcome of the clinical trial based on the probabilistic model and one or more contribution scores of the plurality of contribution scores, the one or more contribution scores being associated with the one or more optimizable elements; and
outputting, by the one or more processors, the explainable prediction for review by a user.

2. The computer-implemented method of claim 1 further comprising:
obtaining, by the one or more processors, an updated trial configuration vector associated with the clinical trial, the updated trial configuration vector comprising one or more optimized elements based on the explainable prediction; and
determining, by the one or more processors, using the trial outcome predictor, an updated probabilistic model of the outcome of the clinical trial based on the updated trial configuration vector.

3. The computer-implemented method of claim 2 further comprising:
outputting, by the one or more processors, the updated probabilistic model of the outcome of the clinical trial for review by the user.

4. The computer-implemented method of claim 2 further comprising:
determining, using the explainability model, a plurality of updated contribution scores for the updated trial configuration vector based on the updated probabilistic model; and
determining, by the one or more processors, one or more changes to the plurality of contribution scores based on a comparison of the plurality of contribution scores and the plurality of updated contribution scores.

5. The computer-implemented method of claim 4 further comprising:
outputting, the one or more processors, the one or more changes to the plurality of contribution scores for review by a user.

6. The computer-implemented method of claim 1, wherein the trial outcome predictor comprises a model chosen from a list including: a k-nearest neighbor model, a random forest model, an elastic net model, and a support vector machine.

7. The computer-implemented method of claim 1, wherein the trial outcome predictor comprises an ensemble model.

8. The computer-implemented method of claim 1, wherein the explainable prediction further comprises one or more further contribution scores associated with the one or more fixed elements.

9. The computer-implemented method of claim 1, wherein the trial configuration vector comprises one or more elements associated with one or more biological features, wherein the one or more biological features are related to a target associated with the clinical trial.

10. The computer-implemented method of claim 9, wherein the one or more biological features comprise at least one hierarchical mechanism of action feature.

11. The computer-implemented method of claim 1, wherein the trial configuration vector comprises one or more elements associated with one or more chemical features, wherein the one or more chemical features are related to a target associated with the clinical trial.

12. The computer-implemented method of claim 1, wherein the trial configuration vector comprises one or more elements associated with one or more design and operation features of the clinical trial.

13. The computer-implemented method of claim 12, wherein the one or more design and operation features include one or more geographical features related to a site associated with the clinical trial.

14. The computer-implemented method of claim 12, wherein the one or more design and operation features include one or more sponsor features related to a sponsor associated with the clinical trial.

15. The computer-implemented method of claim 12, wherein the one or more design and operation features include one or more investigator features related to an investigator associated with the clinical trial.

16. The computer-implemented method of claim 1, wherein the trial configuration vector comprises one or more elements associated with keywords associated with the clinical trial.

17. The computer-implemented method of claim 1, wherein the explainable prediction is included in a report such that the report is output for review by a user.

18. The computer-implemented method of claim 1, wherein the probabilistic model of the outcome of the clinical trial comprises a probability score associated with the outcome of the clinical trial.

19. The computer-implemented method of claim 18, wherein the probability score comprises a probability of the clinical trial moving from a first phase to a second phase.

20. The computer-implemented method of claim 18, wherein the probability score comprises a probability of the clinical trial moving from a second phase to a third phase.

21. The computer-implemented method of claim 18, wherein the probability score comprises a probability of a severe adverse event occurring as part of the clinical trial.

22. The computer-implemented method of claim 18, wherein the probabilistic model of the outcome of the clinical trial further comprises an uncertainty estimate.

23. The computer-implemented method of claim 1, wherein obtaining the trial configuration vector from the one or more data sources comprises generating the trial configuration vector from the one or more data sources.

24. A non-transitory machine readable medium storing instructions for generating an explainable prediction of a trial outcome of a clinical trial, the instructions which, when executed by one or more processors, cause the one or more processors to:
obtain from one or more data sources in communication with the one or more processors, a trial configuration vector associated with the clinical trial, the trial configuration vector comprising one or more fixed elements and one or more optimizable elements,
determine using a trial outcome predictor, a probabilistic model of an outcome of the clinical trial based on the trial configuration vector, the trial outcome predictor having been trained on data related to a plurality of historical clinical trials,
determine using an explainability model, a plurality of contribution scores for the trial configuration vector based on the probabilistic model, each contribution score of the plurality of contribution scores being indicative of a relative contribution of an associated element of the trial configuration vector to the outcome of the clinical trial,
generate an explainable prediction of the trial outcome of the clinical trial based on the probabilistic model and one or more contribution scores of the plurality of contribution scores, the one or more contribution scores being associated with the one or more optimizable elements, and
output the explainable prediction for review by a user.

25. A system configured to generate an explainable prediction of a trial outcome of a clinical trial, the system comprising:
one or more processors; and
a memory storing instructions which, when executed by the one or more processors, cause the one or more processors to:
obtain from one or more data sources in communication with the one or more processors, a trial configuration vector associated with the clinical trial, the trial configuration vector comprising one or more fixed elements and one or more optimizable elements,
determine using a trial outcome predictor, a probabilistic model of an outcome of the clinical trial based on the trial configuration vector, the trial outcome predictor having been trained on data related to a plurality of historical clinical trials,
determine using an explainability model, a plurality of contribution scores for the trial configuration vector based on the probabilistic model, each contribution score of the plurality of contribution scores being indicative of a relative contribution of an associated element of the trial configuration vector to the outcome of the clinical trial,
generate an explainable prediction of the trial outcome of the clinical trial based on the probabilistic model and one or more contribution scores of the plurality of contribution scores, the one or more contribution scores being associated with the one or more optimizable elements, and
output the explainable prediction for review by a user.

26. A computer-implemented method for optimizing parameters of a clinical trial, the method comprising:
obtaining, by one or more processors, from one or more data sources in communication with the one or more processors, a first trial configuration associated with the clinical trial, the first trial configuration comprising values associated with one or more fixed trial parameters and at least one optimizable trial parameter;
obtaining, by the one or more processors, an outcome predictor, wherein the outcome predictor estimates a relationship between a trial configuration of a clinical trial and an outcome of the clinical trial;
optimizing, by the one or more processors, the first trial configuration to improve an outcome of the clinical trial, wherein the step of optimizing comprises:
determining, by the one or more processors, using the outcome predictor and the first trial configuration, an updated value of the at least one optimizable trial parameter such that a first estimated outcome of the clinical trial is greater than a second estimated outcome of the clinical trial; and
creating, the one or more processors, an updated trial configuration comprising the updated value of the at least one optimizable trial parameter;
wherein the first estimated outcome is determined from the outcome predictor based on the updated trial configuration and the second estimated outcome is determined from the outcome predictor based on the first trial configuration; and
outputting, by the one or more processors, the updated trial configuration for review by a user.

27. The computer-implemented method of claim 26 wherein the step of outputting comprises:
generating, by the one or more processors, a report comprising one or more values of the updated trial configuration; and
transmitting, by the one or more processors, the report for display to a user.

28. The computer-implemented method of claim 27 further comprising:
determining, using an explainability model, a first plurality of contribution scores for the updated trial configuration, each contribution score of the first plurality of contribution scores being indicative of a relative contribution of an associated value of the updated trial configuration to the first estimated outcome.

29. The computer-implemented method of claim 28, wherein the report further comprises one or more of the first plurality of contribution scores for the updated trial configuration.

30. The computer-implemented method of claim 28 further comprising:
determining, using the explainability model, a second plurality of contributions scores for the first trial configuration, each contribution score of the first plurality of contribution scores being indicative of a relative contribution of an associated value of the first trial configuration to the second estimated outcome.

31. The computer-implemented method of claim 30, wherein the report further comprises one or more of the second plurality of contribution scores for the first trial configuration.

32. The computer-implemented method of claim 30, wherein the report further comprises a comparison of the first plurality of contribution scores for the updated trial configuration and the second plurality of contribution scores for the first trial configuration.

33. The computer-implemented method of claim 26, wherein the outcome predictor comprises a causal model.

34. The computer-implemented method of claim 33, wherein the relationship determined by the outcome predictor between the trial configuration of the clinical trial and the outcome of the clinical trial comprises a causal relationship determined by the causal model.

35. The computer-implemented method of claim 26, wherein obtaining the first trial configuration from the one or more data sources comprises generating the first trial configuration based on the one or more data sources.

36. A non-transitory machine readable medium storing instructions for optimizing parameters of a clinical trial, the instructions which, when executed by a device comprising one or more processors, cause the one or more processors to:
obtain from one or more data sources in communication with the device, a first trial configuration associated with the clinical trial, the first trial configuration comprising values associated with one or more fixed trial parameters and at least one optimizable trial parameter;
obtain an outcome predictor, wherein the outcome predictor estimates a relationship between a trial configuration of a clinical trial and an outcome of the clinical trial;
optimize the first trial configuration to improve an outcome of the clinical trial, wherein the step of optimizing comprises:
determining using the outcome predictor and the first trial configuration, an updated value of the at least one optimizable trial parameter such that a first estimated outcome of the clinical trial is greater than a second estimated outcome of the clinical trial; and
creating an updated trial configuration comprising the updated value of the at least one optimizable trial parameter;
wherein the first estimated outcome is determined from the outcome predictor based on the updated trial configuration and the second estimated outcome is determined from the outcome predictor based on the first trial configuration; and
output the updated trial configuration for review by a user.

37. A system configured to optimize parameters of a clinical trial, the system comprising:
one or more processors; and
a memory storing instructions which, when executed by the one or more processors, cause the one or more processor to:
obtain from one or more data sources in communication with the one or more processors, a first trial configuration associated with the clinical trial, the first trial configuration comprising values associated with one or more fixed trial parameters and at least one optimizable trial parameter;
obtain an outcome predictor, wherein the outcome predictor estimates a relationship between a trial configuration of a clinical trial and an outcome of the clinical trial;
optimize the first trial configuration to improve an outcome of the clinical trial, wherein the step of optimizing comprises:
determining using the outcome predictor and the first trial configuration, an updated value of the at least one optimizable trial parameter such that a first estimated outcome of the clinical trial is greater than a second estimated outcome of the clinical trial; and
creating an updated trial configuration comprising the updated value of the at least one optimizable trial parameter;
wherein the first estimated outcome is determined from the outcome predictor based on the updated trial configuration and the second estimated outcome is determined from the outcome predictor based on the first trial configuration; and
output the updated trial configuration for review by a user.
